(19) United States
(12) Patent Application Publication
(10) Pub. No.: US 2006/0074323 A1
(43) Pub. Date: Apr. 6, 2006

(54) Display and warning method and apparatus of electronic manometer

(76) Inventor: Yu-Chen Shu, Taipei (TW)

Correspondence Address: Yi-Wen Tseng, 4331 Stevens Battle Lane, Fairfax, VA, 22033, US

(21) Appl. No.: 10/957,584

(22) Filed: Oct. 5, 2004

(51) Int. Cl. A61B 5/02

(52) U.S. Cl. 600/485

(57) ABSTRACT

A method and an apparatus for display the stage of a measured blood pressure of a patient. The blood pressure can be distributed in one of a normal stage, a potential hypertension stage, a first hypertension stage, and a second hypertension stage. When the blood pressure is measured, it is compared to the reference pressure values of various stages by a determination device, so as to determine the specific stage the measured blood pressure belong to. The stage of the blood pressure is then represented by a predetermined number of flashing blocks and/or a predetermined stage symbol.

### BACKGROUND OF THE INVENTION

The present invention relates in general to display and warning method and apparatus for a manometer, and more particularly, to a method and an apparatus which determine which stage group does a measured blood pressure belong to.

Currently, most of electronic manometers provide the measured blood pressure by directly displaying the magnitude thereof. In the Japanese published Application No. 7-31594 and No. 2004-121632, warning functions of electronic manometers have been disclosed.

In the Japanese published application, the electronic manometer is operative to generate a warning signal when the measured blood pressure falls beyond the standard blood pressure range. The standard blood pressure range is determined by the user. Therefore, depending on the professional knowledge of the user, the standard blood pressure range may or may not be a good reference for determining the condition of the measured blood pressure. Further, the warning signal only allows the user to realize whether the measured blood pressure exceeds the standard range or not. Whether a special care or treatment is required cannot be determined from such warning signal.

The Patent No. 2004-121632 classifies the measured blood pressure and displays the classified measured blood pressure in a bar chart. Thereby, warning signal in accordance with the distribution can be generated and displayed. However, such type of manometer is not portable because it requires a large display area and a large volume. In addition, the fabrication cost is relatively high. Further, what each distribution represents has to be printed on the enclosure of the manometer or displayed on the display. Otherwise, the user cannot understand what each bar means.

### BRIEF SUMMARY OF THE INVENTION

A method and an apparatus for display the stage of a measured blood pressure of a patient are provided. The stage includes one of a normal stage, a potential hypertension stage, a first hypertension stage, and a second hypertension stage. When the blood pressure is measured, it is compared to the reference pressure values of various stages by a determination device, so as to determine the specific stage the measured blood pressure belong to. The stage of the blood pressure is then represented by a predetermined number of flashing blocks and/or a predetermined stage symbol.

Preferably, a sound generating device and/or a vibration generating device are provided to generate specific sound and vibration pattern to represent the stage of the blood pressure. The blocks may be configured into various formats, includes an annular ring and a circle divided into a plurality of segments, a plurality of concentric triangular or circular rings, for example.

### BRIEF DESCRIPTION OF THE DRAWINGS

The above objects and advantages of the present invention will be become more apparent by describing in detail exemplary embodiments thereof with reference to the attached drawings in which:

FIG. 1 shows the process for determining the stage of the blood pressure;

FIG. 2 is a table showing the standard critical blood pressure of each stage;

FIG. 3 is circuit diagram of an electronic manometer;

FIG. 4 shows the blood pressure stage determination process;

FIG. 5 shows the display of the electronic manometer;

FIG. 6A to 6D shows the patterns to represent various blood pressure stages;

FIG. 7 shows another example of the display of an electronic manometer;

FIG. 8 shows another example of the display of an electronic manometer;

FIG. 9 shows another example of the display of an electronic manometer;

FIG. 10 shows another application of the razor as shown in FIG. 1;

FIG. 11 shows another example of the display of an electronic manometer;

FIG. 12 shows the flashing blocks for providing alarm.

### DETAILED DESCRIPTION OF THE INVENTION

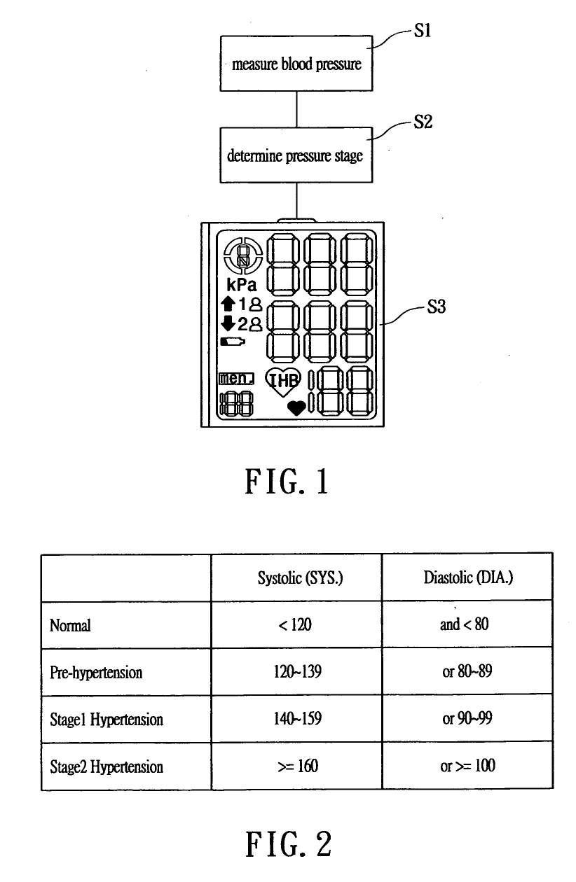

FIG. 1 shows one embodiment of a display of an electronic manometer and a warning method of the electronic manometer. In step S1, a blood pressure is measured by the electronic manometer. In step S2, a determination device is used to determine which stage the measured blood pressure belongs to according to a stage standard. In step S3, stage of the measured blood pressure is represented by flashing blocks, stage symbols, sound or vibration.

By the representation of flashing blocks, stage symbols, sound or vibration, the stage of the measured blood pressure can be cleared provided to the user and/or the patient.

The method for determining the stage of blood pressure adapts the standard published by World Health Organization (WHO). As shown in FIG. 2, the blood pressure can be classified into four stages, including the normal stage, the pre-hypertension stage, the first hypertension stage and the second hypertension stage. The first and second hypertension stages are high risk stages which often cause cardiovascular disease such as heart attack or stroke. The device and method as provided is operative to detect or determine which stage the measured blood pressure belong to, and thus gives a warning or alarm the potential health problem of the patient.

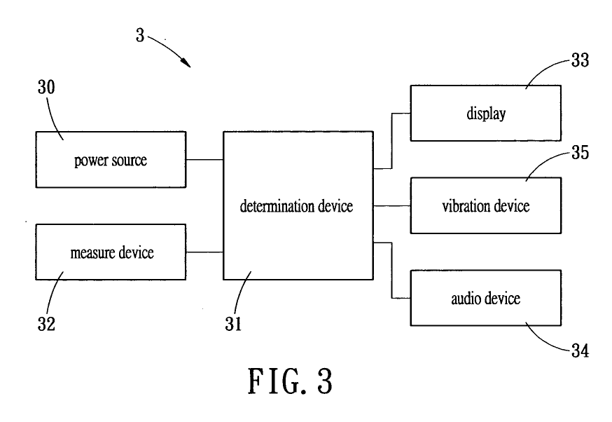

In the embodiment as shown in FIG. 3, the electronic manometer 3 includes an AC or DC power source 30, a measure device 32 for measuring the blood pressure of the user, a determination device 31 in which stages of blood pressure is set up and determined process is installed, and a display 33 to display the stage of the measured blood pressure determined by the determination device 31. The determination result can be represented by flashing blocks 1 and/or stage symbols 2.

The electronic manometer 3 may further comprise an audio device 34 and/or a vibration device 35. The determination device 31 may output a signal to trigger the audio device 34 and/or vibration device 35 according to the determination result. Thereby, the stage of the measured blood pressure determined by the determination device 31 can be represented by the flashing blocks or stage symbols accompanied by sound or vibration.

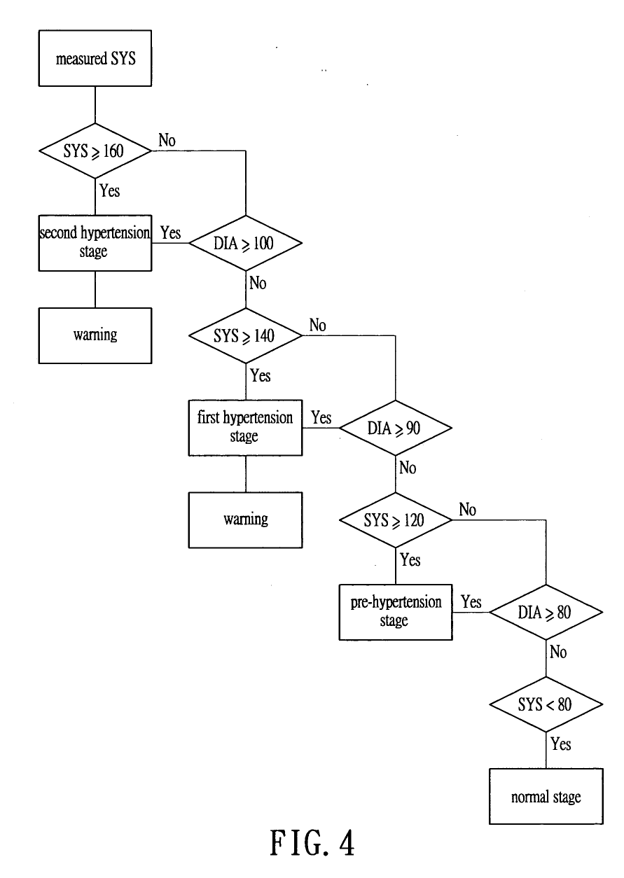

The stage determination process can be referred to FIG. 4. Both systolic pressure and diastolic pressure are used to determine the normal stage, the pre-hypertension stage, the first hypertension stage or the second hypertension stage.

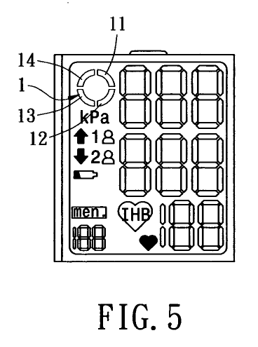

In this embodiment, the measured systolic pressure is used to compare the standard pressure values for each stage. For example, the measured systolic pressure is compared to 160 mmHg for determine whether it falls in the second hypertension stage. If the measured systolic pressure is less than 160 mmHg, the diastolic pressure is compared to 100 mmHg, so as to determine whether the measured diastolic blood pressure falls in the second hypertension stage. If both the measured systolic and diastolic pressures are less than the reference values (160 mmHg and 100 mmHg, respectively), the measured blood pressures are then compared to the reference values of first hypertension stage. Similarly, whether the systolic pressure is over or equal to the 140 mmHg and the diastolic pressure is over or equal to 90 mmHg are determined. If not, the systolic and diastolic pressures are then compared to the reference values 120 mmHg and 80 mmHg, respectively. If the measured systolic and diastolic pressures are over or the same as 120 mmHg and 80 mmHg, respectively, the patient has the risk of potential hypertension. If the systolic and diastolic blood pressures are lower than 120 mmHg and 80 mmHg, respectively, the patient is under normal condition. Whenever the stage of the blood pressure is determined, the comparison stops, and a signal is output to the display for displaying the stage of the blood pressure by flashing block (the reference numeral 1 as shown in FIG. 1) and/or the stage symbol (the reference numeral 2 as shown in FIG. 5). The flashing block and the stage symbol may be accompanied with specific sound or vibration to further distinguish the stages as determined.

The electronic manometer as shown in FIG. 5 includes various stage symbols, including “1” representing the first stage hypertension, “2” representing the second stage hypertension, “P” representing the potential hypertension, and “N” representing normal.

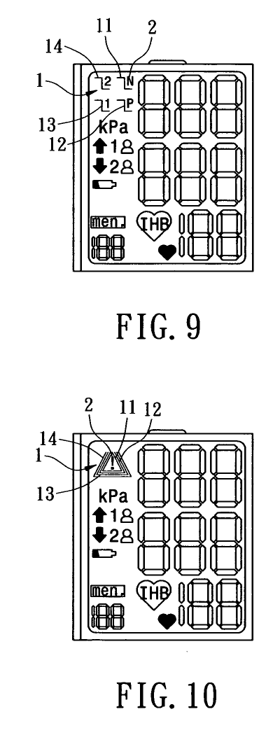

The electronic manometer also includes four blocks 11, 12, 13 and 14. According to the determination result, that is, the stage of the blood pressure as determined, different number of the blocks 11, 12, 13 and 14 will flash as shown in FIGS. 8 and 9.

For example, FIG. 12 shows the display pattern of the first hypertension stage. When the first hypertension stage is determined, the sound generating device 34 may generate a specific sound, and/or the vibration device 35 may be activated to vibrate the manometer to provide the alarm. For different stages of the blood pressure, different numbers of flashing blocks and different patterns of sound and vibration may be generated.

Various types of flashing blocks have been proposed in FIGS. 5-12.

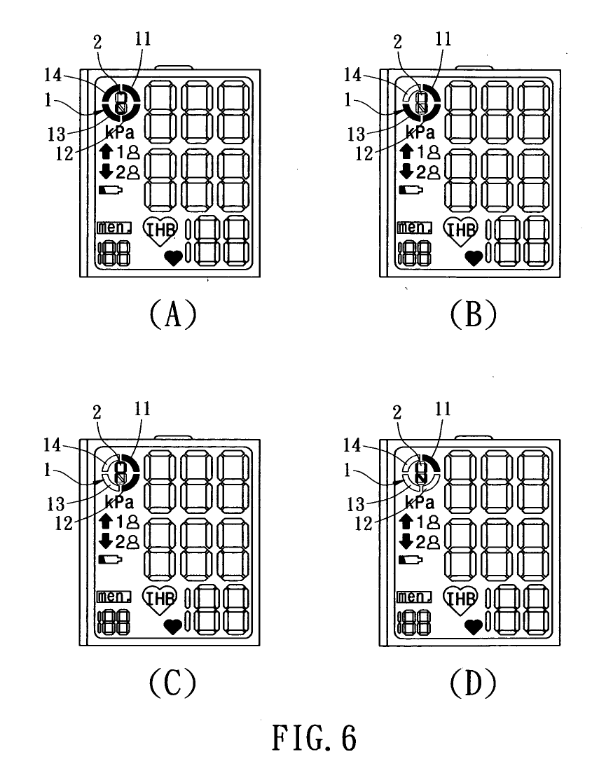

In FIG. 5, an annular area is divided into the first block 11, the second block 12, the third block 13 and the fourth block 14. When the blood pressure is in the second hypertension stage, for example, when the systolic and diastolic pressures are 188 mmHg and 115 mmHg, respectively, all of the first to fourth blocks 11, 12, 13 and 14 are flashing as shown in FIG. 6A. The stage symbol “2” is also switched on. For the first hypertension stage, for example, when the systolic and diastolic pressures are 147 mmHg and 96 mmHg, respectively, the first to third blocks 11, 12 and 13 are flashing, and the stage symbol 1 is on. For the potential hypertension stage, for example, when the systolic and diastolic pressures are 137 mmHg and 86 mmHg, respectively, the first and second blocks 11 and 12 are flashing, and the stage symbol “P” is on. For the normal stage, only the first block 11 is flashing, and the stage symbol “N” is on.

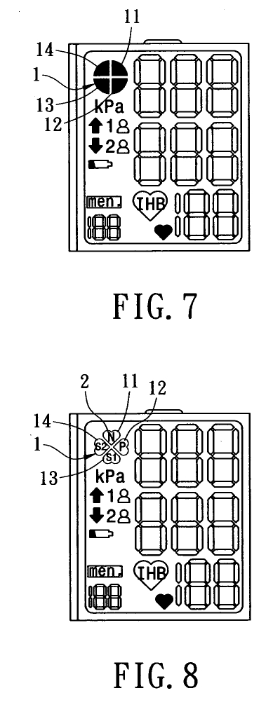

In FIG. 7, a circle is divided into four portions, including the blocks 11, 12, 13 and 14. Again, the stages can be represented by the number of the flashing blocks 11-14 in the similar manner as shown in FIGS. 6A to 6D.

FIG. 8 shows four heart shape blocks 11-14. The number of the flashing blocks 11-14 represents different stages of the blood pressure. Similarly, the flashing blocks can be accompanied with the stage symbols “1”, “2”, “N” and “P”.

In FIG. 9, each of the stage symbols is accompanied with a stair-like flashing block. When any of the specific stage symbols is on to represent one specific stage, the stair-like block is flashing.

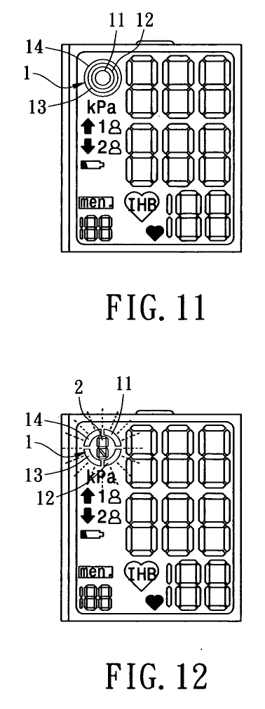

FIG. 10 shows four concentric triangular blocks 11-14, and FIG. 11 shows four concentric annular blocks 11-14. In FIG. 10, an exclamation mark is added at the center of the blocks 11-14. The exclamation mark is lit up or flashing when the blood pressure is in the first or second hypertension stage. In FIG. 11, all the annular blocks 11-14 may be flashing when the blood pressure is in the first hypertension stage. Similar to the embodiment as shown in FIG. 10, an exclamation mark or other warning mark can also be added at the center of the blocks 11-14 to alarm the user the first and second hypertension stages.

While the present invention has been particularly shown and described with reference to preferred embodiments thereof, it will be understood by those of ordinary skill in the art the various changes in form and details may be made therein without departing from the spirit and scope of the present invention as defined by the appended claims.

What is claimed is:

1. A display method of an electronic manometer, comprising:
determining a stage of a measured blood pressure according to a stand reference; and
displaying the stage by flashing a predetermined number of blocks or a stage symbol, or a combination thereof.

2. The method of claim 1, further comprising providing a plurality of blocks operative to selectively flash according to the stage.

3. The method of claim 1, further comprising generating a predetermined sound or a predetermined vibration pattern or a combination there to represent the stage.

4. The method of claim 1, further comprising determining the blood pressure belong to one of a normal stage, a potential hypertension stage, a first hypertension stage and a second hypertension stage.

5. The method of claim 6, further comprising switching a warning mark for the first and second hypertension stages.

6. A warning method for an electronic manometer, comprising:
a) measuring a blood pressure of a patient;
b) determining one of a plurality of stage of the blood pressure;
c) providing a plurality of blocks operative to flash; and
d) selectively flashing the blocks according to the stage of the blood pressure.

7. The method of claim 6, wherein stages include a normal stage, a potential hypertension stage, a first hypertension stage and a second hypertension stage.

8. The method of claim 7, wherein step (c) comprises providing four blocks.

9. The method of claim 7, wherein the blocks are arranged in the form of an annular ring.

10. The method of claim 7, wherein the blocks are configured into four portions of a circle.

11. The method of claim 7, wherein the blocks include four concentric heart-shape blocks.

12. The method of claim 7, wherein the blocks include four concentric triangular rings.

13. The method of claim 7, wherein the blocks include four concentric annular rings.

14. The method of claim 6, further comprising generating a sound according to the stage of the blood pressure.

15. The method of claim 6, further comprising generating a vibration pattern according to the stage of the blood pressure.

16. A warning apparatus for an electronic manometer, comprising:
a device for determining one of a plurality of stage of a measured blood pressure;
a display, comprising:
a plurality of blocks selectively flashing according to the stage of the measured blood pressure; and
a plurality of symbols selectively switched on according to the stage of the measured blood pressure.

17. The apparatus of claim 16, further comprising a sound generating device for generating a predetermined sound of the stage of the measured blood pressure.

18. The apparatus of claim 16, further comprising a vibration generating device for generating a predetermined vibration pattern of the stage of the measured blood pressure.

19. The apparatus of claim 16, wherein the display further comprises a warning mark selectively switched on according to the stage of the blood pressure.

20. The apparatus of claim 16, wherein the stages include a normal stage, a potential hypertension stage, a first hypertension stage and a second hypertension stage.
